(19) United States
(12) Patent Application Publication
(10) Pub. No.: US 2005/0277593 A1
(43) Pub. Date: Dec. 15, 2005

(54) Therapeutic uses of Reg protein

(75) Inventors: Brian K. Dieckgraefe, Chesterfield, MO (US); Anne H. Cross, St. Louis, MO (US)

Correspondence Address: BANNER & WITCOFF, 1001 G STREET N W, SUITE 1100, WASHINGTON, DC, 20001, US

(73) Assignee: Washington University, St. Louis, MO (US)

(21) Appl. No.: 11/133,465

(22) Filed: May 20, 2005

Related U.S. Application Data
(60) Provisional application No. 60/573,340, filed on May 24, 2004.

(51) Int. Cl. A61K 38/17

(52) U.S. Cl. 514/12

(57) ABSTRACT

Human Reg III and Reg IV proteins are used to treat diseases characterized by neurological damage and inflammation. Such diseases include inflammatory bowel disease, sepsis, multiple sclerosis, amyotrophic lateral sclerosis, adrenoleukodystrophy or adrenomyeloneuropathy, spinal cord injury, Devic's disease. Such treatments lead to prevention or amelioration of disease symptoms, and prolongation of life span.

This application claims the benefit of U.S. Ser. No. 60/573,340, filed May 24, 2004, the contents of which are expressly incorporated herein.

The invention was made using funds from the United States government under grant number DK60106 from the National Institutes of Health. Therefore the government retains certain rights in the invention.

### FIELD OF THE INVENTION

The invention relates to therapeutic uses of proteins for treating or preventing neurological or inflammatory diseases.

### BACKGROUND OF THE INVENTION

The human Reg gene family consists of four secreted and structurally unique proteins that share sequence similarity with the carbohydrate-binding domain of C-type lectins. The initial cDNA in this gene family was named Reg for its role in islet of Langerhans regeneration following partial pancreatectomy (now known as Reg la).

Additional members of the human Reg gene family are regenerating gene homologue (Reg Iβ) and pancreatitis-associated protein (Reg III). All are constitutively expressed in the normal proximal gastrointestinal tract. While the function of this gene family is poorly understood, recent data has suggested that Reg family members may function as tissue mitogens. Reg Iα is mitogenic for gastric mucosal cells (1), and pancreatic ductal cells (2,3), and beta cells (4,5). The serum concentration of Reg Iα is significantly increased in many gastrointestinal malignancies, including gastric and pancreatic adenocarcinoma (6). For patients with early stage colonic adenocarcinoma undergoing surgical resection, Reg Iα mRNA expression alone or co-expression of Reg Iα and Reg III mRNA by the carcinoma had a highly significant adverse affect on disease free survival that was independent of tumor stage or site (7). This suggests that expression of members of the Reg protein family may result in a more aggressive tumor phenotype.

We recently identified a novel member of this gene family, Reg IV, which has significant constitutive expression in the distal gastrointestinal tract (8). Molecular modeling of the Reg IV protein showed maintenance of the conserved contact surface residues that cluster on a single face of the 3-dimensional molecule that is present in all other members of the Reg gene family. Nevertheless, other structural differences present in Reg IV suggest that function may not be completely identical to other members of this gene family (8). This protein is of considerable interest because of its possible role in the pathogenesis of colorectal adenocarcinoma. Reg IV is overexpressed in a majority of colorectal adenocarcimonas. By differential display, Reg IV was among several genes with increased mRNA expression in several colon cancer cell lines selected for increased in vitro resistance to cancer chemotherapeutic agents (9).

Regenerating gene (“Reg”) family members are found in the GI system and nervous system of humans and rodents. Rat Reg-2 (equivalent to human Reg III and mouse Reg III beta) is known in the literature to be a potent Schwann cell mitogen and is transported along regenerating axons. Inhibition of Reg-2 signaling retards regeneration (20). Rat Reg 2 has been shown to be up-regulated in regenerating motor and sensory neurons following nerve crush injury (21, 22). Reg-2 expression in motor and sensory neurons during development has been linked to cytokines in the leukemia inhibitory factor (LIF)/ciliary neurotrophic factor (CNTF) family (20), which are produced by astrocytes and are potent survival factors for embryonic and lesioned motoneurons. CNTF and LIF induce rat Reg-2 in cultured motoneurons. Blocking rat Reg-2 expression blocks their neurotrophic effects (23). Thus, Reg-2 is critical in signal transduction for CNTF and LIF. Steroids and IL-6 increase expression of Reg-2 (24).

### BRIEF SUMMARY OF THE INVENTION

In a first embodiment of the invention a method is provided for treating multiple sclerosis. An effective amount of a therapeutic agent is administered to a patient suffering from multiple sclerosis. The therapeutic agent is selected from the group consisting of: human Reg III, human Reg IV protein, and combinations thereof. One or more clinical disease indicators of multiple sclerosis are thereby reduced.

In a second embodiment of the invention a method is provided for treating multiple sclerosis. An effective amount of a therapeutic agent is administered to a patient suffering from multiple sclerosis. The therapeutic agent is selected from the group consisting of: human Reg III, human Reg IV protein, and combinations thereof. The life span of the patient is thereby extended.

In another aspect of the invention a method is provided for treating optic neuritis. An effective amount of a therapeutic agent is administered to a patient suffering from optic neuritis. The therapeutic agent is selected from the group consisting of: human Reg III, human Reg IV protein, and combinations thereof. One or more clinical disease indicators of optic neuritis are thereby reduced.

In another aspect of the invention a method is provided for treating neuromyelitis optica or Devic's disease. An effective amount of a therapeutic agent is administered to a patient suffering from neuromyelitis optica or Devic's disease. The therapeutic agent is selected from the group consisting of: human Reg III, human Reg IV protein, and combinations thereof. One or more clinical disease indicators of neuromyelitis optica or Devic's disease are thereby reduced.

In another aspect of the invention a method is provided for treating adrenoleukodystrophy or adrenomyeloneuropathy. An effective amount of a therapeutic agent is administered to a patient suffering from adrenoleukodystrophy or adrenomyeloneuropathy. The therapeutic agent is selected from the group consisting of: human Reg III, human Reg IV protein, and combinations thereof. One or more clinical disease indicators of adrenoleukodystrophy or adrenomyeloneuropathy are thereby reduced.

In another aspect of the invention a method is provided for treating spinal cord injury.

An effective amount of a therapeutic agent is administered to a patient suffering from spinal cord injury. The therapeutic agent is selected from the group consisting of: human Reg III, human Reg IV protein, and combinations thereof. One or more clinical disease indicators of spinal cord injury are thereby reduced.

In another aspect of the invention a method is provided for treating amyotrophic lateral sclerosis. An effective amount of a therapeutic agent is administered to a patient suffering from amyotrophic lateral sclerosis. The therapeutic agent is selected from the group consisting of: human Reg III, human Reg IV protein, and combinations thereof. One or more clinical disease indicators of amyotrophic lateral sclerosis are thereby reduced.

In another aspect of the invention a method is provided for treating inflammatory bowel disease. An effective amount of a therapeutic agent is administered to a patient suffering from inflammatory bowel disease. The therapeutic agent is selected from the group consisting of: human Reg III, human Reg IV protein, and combinations thereof. One or more clinical disease indicators of inflammatory bowel disease are thereby reduced.

In another aspect of the invention a method is provided for treating or preventing sepsis. An effective amount of a therapeutic agent is administered to a patient suffering from or at risk of developing sepsis. The therapeutic agent is selected from the group consisting of: human Reg III, human Reg IV protein, and combinations thereof. One or more clinical disease indicators of sepsis are thereby reduced or prevented.

### BRIEF DESCRIPTION OF THE DRAWINGS

FIG. 1 shows that treatment with rHuReg III led to EAE amelioration when administered before development of clinical signs, a situation relevant to human MS.

FIG. 2 shows that treatment with rHuReg IV led to EAE amelioration when administered before development of clinical signs, a situation relevant to human MS.

FIG. 3 shows a Kaplan-Meier plot of the death rate in SJL mice treated with placebo (blue) vs low (clear) and high (red)-dose Reg III when treatment was commenced following clinical EAE onset. This situation is more relevant to human MS than treating prior to disease onset. In each of the RegIII treatment groups, only ⅛ mice died of EAE. In the vehicle-treated group, 3/7 mice died due to EAE. One mouse from each group was sacrificed for histologic analyses—thus the “n” of 3 in the vehicle (blue) group and “n” of 6 in the other two groups.

FIG. 4 shows the actual blinded scores for each group described in FIG. 3. The blue group in each figure is the control group. Treatment was commenced after clinical onset of EAE, a situation relevant to human MS.

FIG. 5 shows the sequence of the engineered recombinant Reg IV molecule (SEQ ID NO: 10). The sequence of the expressed protein fusion construct is shown. Bold amino acid residues indicate the engineered human Reg IV sequence. Non-bold residues represent the yeast alpha-factor signal sequence responsible for directing extracellular secretion. Expected Stel3 signal cleavage (filled arrowheads) and Kex2 signal cleavage sites (unfilled arrowhead) are indicated. The location of the amino acid sequences used for the design of synthetic peptides (P4261 and P4262) for generating polyclonal antibodies are shown in brackets.

### DETAILED DESCRIPTION OF THE INVENTION

It is a discovery of the present inventors that human Reg III and Reg IV proteins are able to ameliorate disease activity when administered prophylactically or after the onset of disease symptoms. Disease symptoms which can be ameliorated include those of neurological and inflammatory diseases. In particular, human Reg III and Reg IV proteins can be used to treat or prevent multiple sclerosis, sepsis, adrenoleukodystrophy or adrenomyeloneuropathy, amyotrophic lateral sclerosis, spinal cord injury, inflammatory bowel disease, optic neuritis, Alzheimer's disease, dementia, vasculitis, and ischemic insult.

The Reg proteins have multiple properties that may account for their beneficial effects on these diseases. The invention, however, is not limited to any particular mechanism of action. The Reg proteins stimulate cell survival pathways that may act to reduce axonal and/or neuronal loss. The Reg proteins have direct axonal regenerative properties. The Reg proteins have mitogenic action on Schwann cells and potentially their CNS counterparts, oligodendrogilial cells. The Reg proteins have anti-inflammatory properties. The Reg proteins can cross the blood brain barrier. Members of the Reg gene family of proteins may up-regulate other members of the Reg gene family. Any one or more of these properties may be responsible for the beneficial effects observed in animal models of disease.

Proteins administered are preferably human proteins. They can be obtained from human cells in culture or from cadavers or donated organs. The proteins can also be made recombinantly in cultured cells. The cells can be human, other mammalian, insect, yeast, bacterial, viral, or plant. One particularly suitable yeast host is Pichia pastoris. The proteins can be made in transgenic animals or plants. Human Reg III has been reported to have the sequence described in Genbank Accession no. NP_{—}002571 (SEQ ID NO: 1). Human Reg IV has been reported to have the sequence described in Genbank Accession no. AAG02562 (SEQ ID NO: 2). These sequences represent one allelic version of the proteins. Other allelic versions as are found in the human population can also be used without limitation. Typically these are greater than 95%, greater than 96%, greater than 97%, greater than 98%, greater than 99%, identical to the referenced sequence. Typically these comprise minimal changes such as one or two non-conservative or conservative single amino acid substitutions. Other splice variants may also exist for these proteins and these can be used as well.

Any means known in the art can be used to deliver the therapeutic proteins to patients. These include without limitation the following modes of delivery: intravenous, intrathecal, intranasally, transdernal, subcutaneous, intramuscular, oral, and parenteral. Formulations of the protein will be suitable for administration to a human. Typically such formulations are sterile and pyrogen-free. Buffers and salts may also be used. Other therapeutic agents may be formulated with the Reg III and Reg IV proteins. Other delivery facilitators or stabilizers may also be present. Dosages will depend on the route of administration, the severity of the disease condition, the weight of the patient, and other factors typically considered in tailoring a dose to a patient. Suitable ranges for administration are 0.01 mg/kg to 50 mg/kg, 0.1 mg/kg to 10 mg/kg, and 0.5 mg/kg to 5 mg/kg.

Disease indicators of multiple sclerosis which may be ameliorated or prevented by the treatments with Reg III and/or Reg IV are those that are known in the medical arts. These include without limitation: fatigue, difficulty of walking, bowel and/or bladder disturbance, visual problems, change in cognitive function, abnormal sensation, decreased strength, decreased coordination, weakness or paralysis, change in sexual function, pain, depression and/or mood swings, and associated clinical sequela. Extension of expected life span due to multiple sclerosis may also be observed as a result of the treatments with Reg III and/or Reg IV.

Other inflammatory or neurological diseases which can be effectively treated with Reg III and Reg IV proteins include optic neuritis, neuromyelitis optica or Devic's disease, adrenoleukodystrophy or adrenomyeloneuropathy, spinal cord injury, amyotrophic lateral sclerosis, inflammatory bowel disease, and sepsis. Symptoms of these diseases can be ameliorated by the treatments. In some cases disease can be prevented if patients at risk are treated. Disease-induced death can be delayed or prevented. For example, patients at risk of developing sepsis can be treated to prevent or delay onset of sepsis or ameliorate its severity.

Disease indicators of inflammatory bowel disease include diarrhea, fever, abdominal pain, anemia, tenesmus, loss of appetite, weight loss, weakness, fluid and electrolyte losses, fistulas. Disease indicators of sepsis include fever, hypothermia, hyperventilation, chills, shaking, warm skin, skin rash, tachycardia, delirium, decreased urine output. Disease indicators of optic neuritis include blurring of vision, loss of visual acuity, loss of color vision, complete or partial blindness, and pain behind the eye. Disease indicators of neuromyelitis optica include optic neuritis, numbness, muscle weakness, spasticity, ataxia, uncoordination, and myelitis. Disease indicators of adrenoleukodystrophy include behavioral changes such as abnormal withdrawal or aggression, poor memory, and poor school performance. Other symptoms include visual loss, learning disabilities, seizures, poorly articulated speech, difficulty swallowing, deafness, disturbances of gait and coordination, fatigue, intermittent vomiting, increased skin pigmentation, and progressive dementia, progressive stiffness, weakness or paralysis of the lower limbs, and ataxia. Disease indicators of adrenomyeloneuropathy include gait disturbance, sensory disturbance, decreased strength, decreased dexterity, stiffness, bladder dysfyunction, increased skin pigmention, and ataxia. Disease indicators of spinal cord injury include paralysis (loss of control over voluntary movement and muscles of the body) and loss of sensation and reflex function below the point of injury, including autonomic activity such as breathing and other activities such as bowel and bladder control, pain or sensitivity to stimuli, muscle spasms, and sexual dysfunction. Disease indicators of amyotrophic lateral sclerosis include tripping and falling, loss of motor control in hands and arms, dysarthia, spasticity, hyperflexia, fasciculations, dysphagia, difficulty speaking, swallowing and/or breathing, persistent fatigue, and twitching and cramping. Other relevant disease indicators or symptoms may be monitored and ameliorated or prevented.

### EXAMPLES

We have tested Reg III treatment in both the “preventative” and “therapy” modes against experimental autoimmune encephalomyelitis (EAE) in SJL mice. Endpoints assessed blindedly were clinical disease, and CNS expression (using real-time PCR) of several myelin, neuronal, axonal, and CNS growth factor genes selected based upon preliminary exploratory DNA microarrays.

### Example 1

Microarray studies on EAE in mice.

We used real-time PCR to determine if the Reg proteins are present or increased in expression during EAE in mice. Reg gene expression (mRNA) in CNS tissues of EAE-affected SJL female mice (2 each at onset, peak disease, and relapse), naïve controls (n=2) and mice immunized with CFA alone (n=2) were analyzed relative to the housekeeping genes actin and GAPDH. Up-regulation of mRNA including mouse Reg 1117, which has homology with HuReg III and rat Reg-2, occurred during EAE.

### Example 2

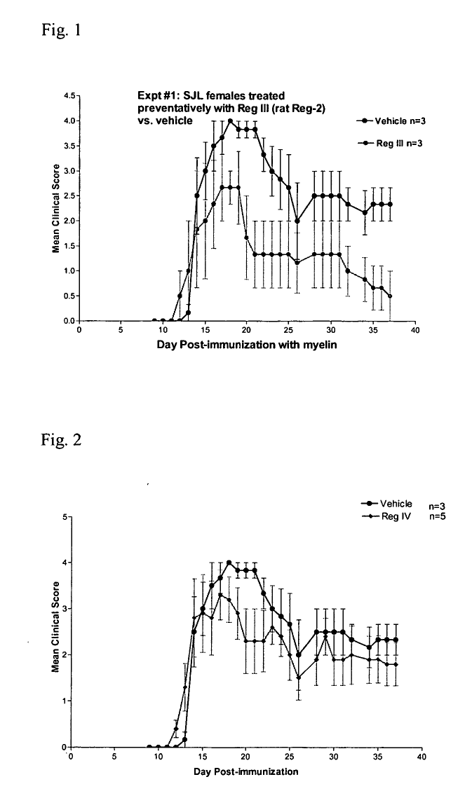

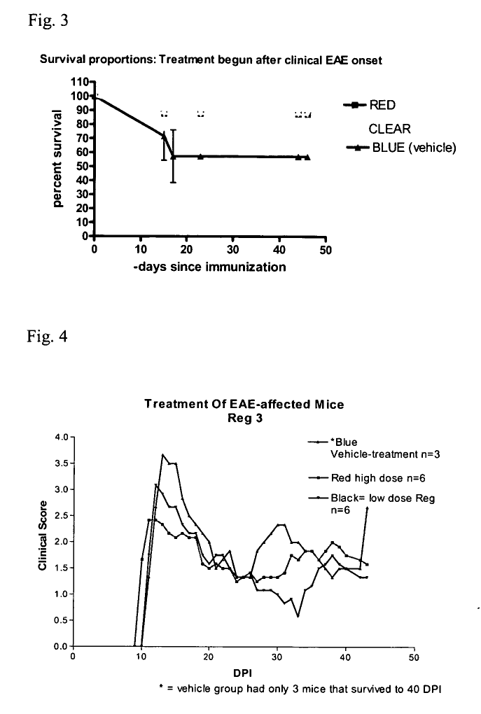

Recombinant Human (rHu) Reg proteins were tested for therapeutic efficacy in SJL murine EAE. Both rHuReg III and IV ameliorated disease severity when used preventatively. Mice were scored blindedly using an ordered categorical scale (clinical scoring of 0 to 5; Ref.7). Comparisons between groups utilize nonparametric tests. For preventative treatment the difference between HuReg III and vehicle was significant using the 2-tailed Mann-Whitney test (p<0.0006), as was Hu Reg IV-vs. placebo-treated mice (p<0.027, FIGS. 1 and 2). Although mathematically inaccurate, means and standard errors of EAE scoring are often used to visually depict EAE experimental scores. FIG. 1 below shows the daily mean scores +/−SEM for Reg III vs. vehicle-treatment used in preventative fashion. Vehicle-treated mice are the control mice.

### Example 3

Gene expression in CNS of mice from the first treatment experiment was analyzed using oligonucleotide-based microarrays. RNA was extracted from spinal cords of placebo- and HuReg-III-treated mice following perfusion with RNAse-free saline. Microarrays were performed in duplicate. Only genes showing >1.6 log-fold up-regulation or down-regulation in comparison to controls in both duplicates were considered further. Several interesting genes involved in CNS growth were up-regulated in mice treated with Reg III, including myelin oligodendrocyte basic protein (MOBP), transthyretin, FGF-1, Fibulin 5 (neural crest EGF-like protein), DIPB gene (expressed in developing neuroblasts). Several inflammatory genes were down-regulated including MMP-3, Mouse class II transactivator C2ta, TNF receptor associated factor 2 (TRAF2), and several Vbeta TCR genes. Up-regulated growth genes and down-regulated inflammatory genes may be a direct drug effect or an indirect effect due to less inflammation and pathology in Reg-III and Reg-IV treated mice.

### Example 4

Expression of Recombinant Human Regenerating Gene IV in P. pastoris.

The cDNA corresponding to mature human Reg IV was fused in frame with the Saccharomyces cerevisiae α-mating factor secretion signal in the vector pPICZα (Invitrogen Corporation, Carlsbad, Calif.). This construct provides antibiotic selection in both E. coli and P. pastoris and directs the synthesis and extracellular secretion of Reg IV coupled with cleavage of the amino-terminal secretion sequence. This fusion protein is placed under the control of the Pichia AOX1 methanol-inducible promoter. Sites of predicted Kex2 and Stel3 cleavage are shown. As a result of our cloning strategy, two additional amino acids, Glu-Phe, follow the second Stel3 cleavage site, and precede the Asp-Ile-Ile-Met (SEQ ID NO: 3) residues that initiate the amino terminus of mature Reg IV. Structural homology between Glu-Ala, a known Stel3 site, and Glu-Phe, raised the possibility that Stel3 might also recognize this as an alternative cleavage site. Approximately 300 Zeocin resistant transformants of KM71H resulted from electroporation, which were of the expected Mut^{S} phenotype. Twenty-five were randomly selected and screened for functional expression of the Reg IV protein by SDS-PAGE and Coomassie staining. Aliquots of the cleared culture supernatant were examined to identify a single clone with the highest level of Reg IV protein production.

Fermentation and Characterization of Recombinant hReg IV in P. pastoris.

The P. pastoris expression clone selected above was used for production of human Reg IV in shaker flask culture (Pichia Manual, Invitrogen Corporation, Carlsbad, Calif.). Yields of protein were approximately 1-2 mg per liter of starting culture, disappointingly low for planned animal studies (data not shown). Accordingly, we attempted to scale up production in both yield and volume by fermentation. Fermentation of the clone expressing hReg IV was carried out using a BioFlo 110 fermenter (New Brunswick Scientific Co.) equipped with software control of the growth conditions and data logging. An overnight broth of KM71H containing the expression construct hReg IV-pPICZα was used to inoculate 3.5 liters of fermentation basal salt-glycerol media in a 5.6 liter working volume fermenter. The initial batch phase growth occurred in media containing 40 gm/liter glycerol. Following a marked rise in dO2 occurring as a result of carbon source limitation at approximately 26 hrs, the glycerol fed-batch process was initiated. Glycerol was provided as a 50% glycerol solution containing 12 mL/liter of the trace minerals solution, administered at 40 mLs/hr for 14 hours to induce rapid expansion of the Pichia cell mass. The production phase was initiated by methanol feeding after a 3 hr carbon-source starvation. The rate of methanol feeding was slowly ramped up over 8 hours as described in methods and continued until 134 hours. The software control was set to maintain dO2≧30% during growth on glycerol and ≧25% during growth on methanol, requiring administration of supplemental oxygen between 28-42 hrs and between 58-103 hrs. Although not a part of a regular production batch, in some runs the methanol feeding was discontinued at 134 hours to assess the time until depletion of the carbon source, by dO2 monitoring. Typically, the dO2 would rise to ≧100% within 2-3 hrs. At the initiation of the 96 hour production phase, and again after 24 hours, 10 grams of casamino acids were added as a supplemental nitrogen source and to minimize the activity of yeast produced proteases. During the production phase, the temperature of the reaction chamber was reduced to 26° C. Fermentation runs performed without a reduction in the reaction chamber temperature or without the addition of casamino acids had appreciably less of the Reg IV product running at ˜15-16 kDa in association with the prominent appearance of lower molecular weight proteins of approximately 8 and 12 kDa molecular weight by SDS-PAGE. Amino terminus sequencing of these lower bands revealed the expected N-terminus of Reg IV, demonstrating that these lower molecular weight bands resulted from internal proteolysis near the carboxy terminus.

The production of recombinant human Reg IV was monitored by SDS-PAGE and Coomassie blue staining. Samples taken at approximately 24 and 48 hrs, during growth on glycerol, lacked significant expression of any secreted protein. Whereas samples taken at 72, 96, 120, and 144 hrs, all times following activation of the alcohol oxidase (AOX1) promoter, had a prominent band at ˜15 kDa, corresponding to the expected size of human Reg IV. To determine the identity of this band, Western blotting was performed using the monoclonal and two polyclonal antibodies generated as described in methods. Bands were visualized by enhanced chemiluminescence (ECL, Amersham). Identical staining was demonstrated using anti-P4262 and anti-P4261, polyclonal antibodies raised against 15-mer synthetic peptides representing two non-overlapping sequences derived from the human Reg IV cDNA sequence. Purification steps.

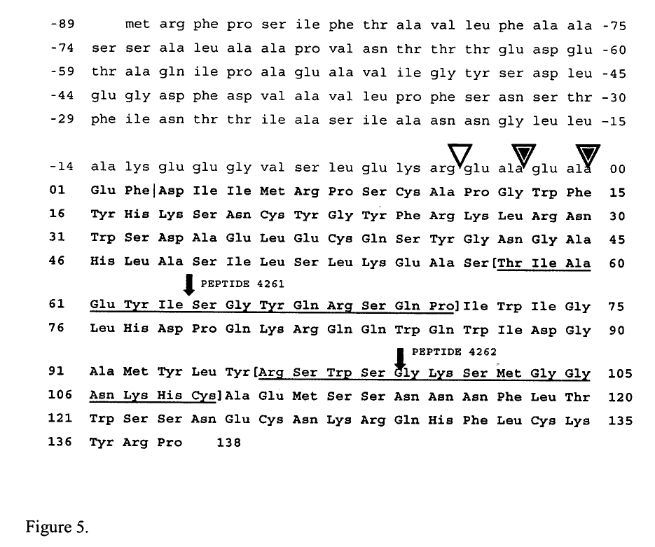

Recombinant human Reg IV was initially purified by tangential flow filtration. As is typical for the methylotrophic yeast Pichia pastoris, cleared supernatants are pale green in color. The centrifuged fermentation supernatant (˜3500 mL) was initially cleared using a 0.1 square meter Durapore 0.45 μM PVDF cartridge. Proteins or protein aggregates greater than 50 kDa were discarded in the Biomax 50 kDa polyethersulfone cartridge retentate. The substance resulting in the green color of the media was also removed in this step. A 50 kDa cutoff was selected because of the paucity of protein contaminants below this mass, and the well described propensity for other Reg family members to undergo dimerization (11). Seventy percent of the Reg IV was recovered in the 50 kDa permeate. Contaminants less than 8 kDa were then removed in the discarded 8 kDa permeate. Overall, thirty-five percent of the original Reg IV was recovered in the 8 kDa retentate (˜250 mLs). Higher yields could be obtained by greater flushing of the 50 and 8 kDa filter cassettes, but this comes at the price of a significantly larger dilution of the final protein solution. The final purification step involved C₁₈RP-HPLC. Differences in the retention time as well as the broad elution profile for the fermentation sample suggested that there could be differences in the final protein products. We estimated an overall yield of about 25-28% of the original Reg IV. Individual bands were subjected to Edman N-terminal sequence analysis on a PE-Biosystems Procise ABI494 (Foster City, Calif.). Unexpectedly, the processing and secretion of Reg IV differed between shaker flask production and fermentation. Furthermore, the leading shoulder for each preparation contained a prominent higher molecular weight band in addition to the expected Reg IV product. The sequence of the engineered recombinant Reg IV molecule is shown in FIG. 5. Edman N-terminal sequence of Reg IV produced by batch growth in a shaker flask was the DIIMRPSC (SEQ ID NO: 7), the expected amino terminus of mature Reg IV. The higher molecular weight band gave an identical sequence suggesting that this is a dimeric species of Reg IV that has survived both C₁₈RP-HPLC and heat treatment at 95 degrees C. for 5 minutes in the presence of SDS and DTT prior to SDS-PAGE analysis. The N-terminal sequence of Reg IV produced under fermentation conditions was EFDIIMRPSC (SEQ ID NO: 8), reflecting inclusion of the two additional amino acids, Glu-Phe, that follow the second Stel3 cleavage site (filled arrowhead). A similar dimeric species containing the extended N-terminus was observed for the fermentation sample, yet tended to “bleed-across” the entire peak unlike the shaker flask counterpart. This data highlights the potential for differences in the processing and secretion of recombinant Reg IV unique to specific Pichia growth conditions. MALDI-TOFMS of the Reg IV produced by batch growth in a shaker flask showed a MWₑₓₚₜ. of 15,917.3 in good agreement with an expected unmodified MW_{calc}. of 15,913.9. MALDI-TOFMS for Reg IV produced by fermentation showed a MWₑₓₚₜ. of 16,192.7, in agreement with a MW_{calc}. of 16,190.2, corresponding to the amino terminus starting with EFDIIMR (SEQ ID NO: 9).

Vectors, Strains and Supplies: The pPICZα vector (Invitrogen) was used to direct secretion of human Reg IV. The pGEM-T Easy vector (Promega) was used to clone and sequence the PCR product. Escherichia coli DH5α was used for subcloning and Reg IV-pPICZα vector cloning. The Pichia pastoris strain KM71H (genotype, arg4 aox1::ARG4) was used for Reg IV protein expression. Preparative-scale (Pellicon 2) Biomax polyethersulfone cartridges with an 8 kDa and 50 kDa molecular weight cutoff and Durapore cartridges with a 0.45 μM pore size were purchased from Millipore. Antifoam 204 (Sigma) was autoclaved and used at a 1:50 dilution. Components of the trace mineral solution [Fe₂(SO₄)-7H₂O, 65 gm/liter; ZnSO₄, 42.2 gm/liter; CuSO₄-5H₂O, 6 gm/liter; MnSO₄—H₂O, 3 gm/liter; CoCl₂-6H₂O, 0.5 gm/liter; Na₂MoO₄-2H₂O, 0.2 gm/liter; NaI, 0.08 gm/liter; H₃BO₃, 0.02 gm/liter] were purchased from Sigma.

Construction of the human regenerating gene IV expression vector: The cloning and cDNA sequence for human Reg IV has been previously reported (8). The cDNA encoding the mature secreted form of Reg IV, lacking the signal sequence, was amplified by PCR using the specific primers 5′-GGAATTCGATATCATCATGAGACCCAGCTG-3′ (SEQ ID NO: 4) and 5′-CTAACTCCTGCACAGCCCCGTCCTCTAGAGG-3′ (SEQ ID NO: 5). The forward primer contained an EcoR I site for cloning into pPICZα. The reverse primer incorporated the native stop codon and an engineered Xba I site. PCR products and vector were digested with EcoR I and Xba I, gel purified and ligated together. E. coli DH5α: was chemically transformed with the recombinant vector and cultured at 37° C. on low-salt LB with Zeocin (25 μg/mL) for selection of recombinants. The recombinant plasmid hReg IV-pPICZα was sequenced to ensure 100% identity with the expected nucleotide sequence and in-frame orientation.

Construction and Isolation of Reg IV expressing P. pastoris clones: The hReg IV-pPICZα plasmid was purified and linearized with Sal I prior to electroporation into P. pastoris KM71H. Yeast transformants were selected on YPD agar plates (2% Bacto peptone, 1% yeast extract, 2% dextrose) containing 1M sorbitol and 100 μg/mL Zeocin. Individual Zeocin resistant clones were screened for protein production in 50 mL culture tubes. Colonies were used to innoculate 5 mLs of BMGY media (2% Bacto peptone, 1% yeast extract, 0.34% yeast nitrogen base, 1% ammonium sulfate, 1% glycerol, 0.4 μg/mL Biotin, buffered with 1/10 volume of pH 6.0 potassium phosphate buffer) and grown with shaking overnight. Subsequently the yeast were collected by centrifugation and resuspended in ¼^{th} of the original culture volume of BMMY media (same as BMGY except same volume of methanol is substituted for glycerol) were cultured for a total of 72 hours at 30° C. with shaking. Cleared supernatants were screened for protein expression by 12% SDS-PAGE and Coomassie brilliant blue R250 staining. Reg IV expressing Pichia clones were suspended in YPD media with 15% glycerol and stored at −80° C. Limited quantities of recombinant Reg IV were produced by direct scale up of these conditions in 2 liter shaker flasks.

Fermentation: Fermentation of the hReg IV (Mut^{s}) transformant of P. pastoris was carried out using a BioFlo 110 fermenter (New Brunswick Scientific Co.) equipped with software control (BioCommand Plus) of temperature (26 and 30° C.), agitation (1000 rpm), pH, anti-foam addition, supplemental oxygen addition (dO₂≧30% and ≧25%) and data logging. A frozen stock of KM71H containing the expression construct Reg IV-pPICZα was used to inoculate a 2 liter baffled flask containing 300 mLs of BMMY media containing 100 μg/mL Zeocin. After overnight incubation, the shaker flask contents were transferred to the 5.6 liter working volume fermenter containing 3.5 liters of fermentation basal salt-glycerol media [H₃PO₄, 27 mL/liter; CaSO₄.2H₂O, 0.9 gm/liter; K₂SO₄, 18 gm/liter; MgSO₄.7H₂O, 15 gm/liter; KOH, 4.1 gm/liter; glycerol 40 gm/liter; D-biotin, 0.87 mg/liter] supplemented with 4.4 mL/liter trace mineral solution. pH was maintained at 5.0 with 28% (w/v) NH₄OH during batch and fed-batch growth on glycerol. Growth in batch mode was continued until the dissolved oxygen concentration increased as a result of carbon source limitation (26 hours). A 50% glycerol feeding solution was initiated (40 mL/hour) for 14 hours and supplemental oxygen automatically administered to maintain the dissolved oxygen concentration above 30%. Three to four hour carbon source starvation followed to ensure complete glycerol consumption and facilitate methanol induction of protein expression. Methanol feeding was initiated (100% methanol containing 12 mL trace minerals/liter) at 4 mL/hour for 2 hours, and the feed rate then increased by 2.7 mLs every 2 hours to a final rate of 12 mL/hour. At the initiation of the 96 hour production phase, and again after 24 hours, 10 grams of casamino acids (DIFCO laboratories) were added as a supplemental nitrogen source and to minimize the activity of yeast produced proteases. During the production phase, the temperature of the reaction chamber was reduced to 26° C. and supplemental oxygen was added as needed to maintain the dO₂≧25%.

Protein Purification and Analysis: Fermentation broth containing the secreted rHuReg IV protein was collected by centrifugation (3500 rpm). The supernatant was processed in a Millipore Pellicon-2 tangential flow filtration apparatus using a PVDF Durapore membrane with a pore size of 0.45 M. The filtrate was retained and processed through a Biomax 50 K polyethersulfone membrane. This filtrate was then processed through a Biomax 8 K polyethersulfone membrane. The final purification step involved reverse phase HPLC of the 8 K retentate solution. rHuReg IV was applied to a C₁₈RP-HPLC column (Vydac 218TP54, The NEST Group, Southborough, Mass.) at 37° C. in 0.1% trifluoracetic acid. Samples were eluted using a linear gradient from 0 to 80% of 0.095% trifluoracetic acid, 90% acetonitrile B in 60′ at 1 mL/min following a 10 min 0% β cratic wash. Selected fractions were analyzed by Matrix Assisted Laser Desorption Ionization Time-of-Flight Mass Spectrometry (MALDI-TOFMS) on an Applied Biosystems Voyager-DE STR instrument (Foster City, Calif.). One Pl of sample matrix (10 mg/mL α-cyano-4-hydroxycinnamic acid in 50% MeCN, 0.1% TFA) was spotted on the stainless steel grid and allowed to air dry. Then, one μl of sample+matrix which was mixed at volume ratios of 1/1 to 1/9 was applied to the grid and air dried. The instrument was operated in the linear, delayed extraction, positive polarity mode using an accelerating voltage of 20,000 V, 93% grid voltage, and 700 nsec extraction delay. External calibration using recombinant human Reg Iα and its tryptic fragment S1 (10) provided an accuracy of about 1 part per 5,000. Additional samples were subjected to SDS-PAGE. Proteins were transferred to PVDF membrane and Coomassie stained. Individual bands were excised and subjected to Edman N-terminal sequence analysis on a PE-Biosystems Procise ABI494 (Foster City, Calif.).

### Example 5

The cDNA sequence encoding human Reg III was similarly cloned into picZalpha and the clone was designed to express the mature protein following cleavage by yeast proteases. The human Reg III sequence, including the signal sequence (indicated by kets), is shown below.

| | |
|---|---|
| 1 | [mlppmalpsv swmllsclml lsqvqg] --eepq relpsarirc pkgskaygsh cyalflspks |
| 61 | wtdadlacqk rpsgnlvsvl sgaegsfvss lvksignsys yvwiglhdpt qgtepngegw |
| 121 | ewsssdvmny fawernpsti sspghcasls rstaflrwkd yncnvrlpyv ckftd. |

(SEQ ID NO: 1.) Following methanol induction in Pichia as detailed example 4, the protein was sequentially purified using tangential flow ultrafiltration and reverse phase HPLC. The N-terminal sequence for C18 reverse phase-HPLC purified sample from a Reg III prep run on SDS-PAGE and transferred to PVDF was: EEPQRELPSARIRCPKGSKA (SEQ ID NO: 6), the exact expected sequence of the mature protein, lacking the signal peptide (indicated by brackets above). The calculated molecular weight (MW calc.) was 16,566.4 kDa, identical to the molecular weight determined experimentally by MALDI-TOF (16,556.5).

### LITERATURE CITED

1. Fukui, H., Kinoshita, Y., Maekawa, T., Okada, A., Waki, S., Hassan, S., Okamoto, H., and Chiba, T. (1998) Regenerating gene protein may mediate gastric mucosal proliferation induced by hypergastrinemia in rats. Gastroenterology 115, 1483-1493
2. Zenilman, M. E., Magnuson, T. H., Perfetti, R., Chen, J., and Shuldiner, A. R. (1997) Pancreatic reg gene expression is inhibited during cellular differentiation. Annals of Surgery 225, 327-332
3. Zenilman, M. E., Perfetti, R., Swinson, K., Magnuson, T., and Shuldiner, A. R. (1996) Pancreatic regeneration (reg) gene expression in a rat model of islet hyperplasia. Surgery 119, 576-584
4. Zenilman, M. E., Magnuson, T. H., Swinson, K., Egan, J., Perfetti, R., and Shuldiner, A. R. (1996) Pancreatic thread protein is mitogenic to pancreatic-derived cells in culture. Gastroenterology 110, 1208-1214
5. Watanabe, T., Yonemura, Y., Yonekura, H., Suzuki, Y., Miyashita, H., Sugiyama, K., Moriizumi, S., Unno, M., Tanaka, O., Kondo, H., and et al. (1994) Pancreatic beta-cell replication and amelioration of surgical diabetes by Reg protein. Proceedings of the National Academy of Sciences of the United States of America 91, 3589-3592
6. Satomura, Y., Sawabu, N., Mouri, I., Yamakawa, O., Watanabe, H., Motoo, Y., Okai, T., Ito, T., Kaneda, K., and Okamoto, H. (1995) Measurement of serum PSP/reg-protein concentration in various diseases with a newly developed enzyme-linked immunosorbent assay. Journal of Gastroenterology 30, 643-650
7. Macadam, R. C., Sarela, A. I., Farmery, S. M., Robinson, P. A., Markham, A. F., and Guillou, P. J. (2000) Death from early colorectal cancer is predicted by the presence of transcripts of the REG gene family. British Journal of Cancer. 83, 188-195
8. Hartupee, J. C., Zhang, H., Bonaldo, M. F., Soares, M. B., and Dieckgraefe, B. K. (2001) Isolation and characterization of a cDNA encoding a novel member of the human regenerating protein family: Reg IV. Biochimica et Biophysica Acta. 1518, 287-293
9. Violette, S., Festor, E., Pandrea-Vasile, I., Mitchell, V., Adida, C., Dussaulx, E., Lacorte, J. M., Chambaz, J., Lacasa, M., and Lesuffleur, T. (2003) Reg IV, a new member of the regenerating gene family, is overexpressed in colorectal carcinomas. International Journal of Cancer. 103, 185-193
10. Dieckgraefe, B. K., Crimmins, D. L., Landt, V., Houchen, C., Anant, S., Porche-Sorbet, R., and Ladenson, J. H. (2002) Expression of the regenerating gene family in inflammatory bowel disease mucosa: Reg Ialpha upregulation, processing, and antiapoptotic activity. Journal of Investigative Medicine. 50, 421-434
11. Bodeker, H., Keim, V., Fiedler, F., Dagorn, J. C., and lovanna, J. L. (1999) PAP I interacts with itself, PAP II, PAP III, and lithostathine/reglalpha. Molecular Cell Biology Research Communications. 2, 150-154
12. Rechreche, H., Montalto, G., Mallo, G. V., Vasseur, S., Marasa, L., Soubeyran, P., Dagorn, J. C., and lovanna, J. L. (1999) pap, reg 1 alpha and reg 1 beta mRNAs are concomitantly up-regulated during human colorectal carcinogenesis. International Journal of Cancer 81, 688-694
13. Kannan, R., Tomasetto, C., Staub, A., Bossenmeyer-Pourie, C., Thim, L., Nielsen, P. F., and R10, M. (2001) Human pS2/trefoil factor 1: production and characterization in Pichia pastoris. Protein Expression & Purification. 21, 92-98
14. Chi, X. S., Landt, Y., Crimmins, D. L., Dieckgraefe, B. K., and Ladenson, J. H. (2002) Isolation and characterization of rabbit single chain antibodies to human Reg Ialpha protein. Journal of Immunological Methods. 266, 197-207
15. Potten, C. S., Wilson, J. W., and Booth, C. (1997) Regulation and significance of apoptosis in the stem cells of the gastrointestinal epithelium. Stem Cells. 15, 82-93
16. Rosty, C., Christa, L., Kuzdzal, S., Baldwin, W. M., Zahurak, M. L., Carnot, F., Chan, D. W., Canto, M., Lillemoe, K. D., Cameron, J. L., Yeo, C. J., Hruban, R. H., and Goggins, M. (2002) Identification of hepatocarcinoma-intestine-pancreas/pancreatitis-associated protein I as a biomarker for pancreatic ductal adenocarcinoma by protein biochip technology. Cancer Research. 62, 1868-1875
17. Harada, K., Zen, Y., Kanemori, Y., Chen, T. C., Chen, M. F., Yeh, T. S., Jan, Y. Y., Masuda, S., Nimura, Y., Takasawa, S., Okamoto, H., and Nakanuma, Y. (2001) Human REG I gene is up-regulated in intrahepatic cholangiocarcinoma and its precursor lesions. Hepatology. 33, 1036-1042
18. Cerwenka, H., Aigner, R., Bacher, H., Werkgartner, G., el-Shabrawi, A., Quehenberger, F., and Mischinger, H. J. (2001) Pancreatitis-associated protein (PAP) in patients with pancreatic cancer. Anticancer Research. 21, 1471-1474
19. Bernard-Perrone, F. R., Renaud, W. P., Guy-Crotte, O. M., Bernard, P., Figarella, C. G., Okamoto, H., Balas, D. C., and Senegas-Balas, F. O. (1999) Expression of REG protein during cell growth and differentiation of two human colon carcinoma cell lines. Journal of Histochemistry & Cytochemistry 47, 863-870
20. Livesey F J, O'Brien J A, Li M, Smith A G, Murphy L J, Hunt S P. A Schwann cell mitogen accompanying regeneration of motor neurons. Nature 390: 614-618, 1997
21. Livesy F J, Hunt S P. Differential Display cloning of genes induced in regenerating neurons. Methods in Enzymology 16: 386-395, 1998
22. Averill S, Davis D R, Shortland P J, Priestley J V, Hunt S P. Dynamic pattern of reg-2 expression in rat sensory neurons after peripheral nerve injury. J. Neurosci. 22(17):7493-501, 2002
23. Nishimune H, Vasseur S, Wiese S, Birling M C, Holtmann B, Sendtner M, lovanna J L, Henderson C E. Reg-2 is a motoneuron neurotrophic factor and a signalling intermediate in the CNTF survival pathway. Nat Cell Biol. December; 2(12):906-14, 2000
24. Dusetti N J, Ortiz E M, Mallo G V, Dagorn J C, lovanna J L. Pancreatitis-associated protein I (PAP I), an acute phase protein induced by cytokines. Identification of two functional interleukin-6 response elements in the rat PAP I promoter region. J. Biol. Chem 270: 22417-22421, 1995
25. Hartupee J C. Zhang H. Bonaldo M F. Soares M B. Dieckgraefe B K. Isolation and characterization of a cDNA encoding a novel member of the human regenerating protein family: Reg IV. Biochimica et Biophysica Acta. 1518(3):287-93, 2001
26. Cross, A. H., Girard, T. J., Giacoletto, K. S., Evans, R. J., Keeling, R. M., Lin, R. F., Trotter, J. L., and Karr, R. W.: Long-term inhibition of murine experimental autoimmune encephalomyelitis using CTLA-4-Fc supports a key role for CD28 costimulation J. Clin. Invest. 95:2783-2789, 1995.

1. A method for treating multiple sclerosis, comprising:
administering to a multiple sclerosis patient an effective amount of a therapeutic agent selected from the group consisting of: human Reg III, human Reg IV protein, and combinations thereof, whereby one or more clinical disease indicators of multiple sclerosis are reduced.

2. A method for treating multiple sclerosis, comprising:
administering to a multiple sclerosis patient an effective amount of a therapeutic agent selected from the group consisting of: human Reg III, human Reg IV protein, and combinations thereof, whereby life span of the patient is extended.

3. The method of claim 1 wherein the human Reg protein is a recombinant protein.

4. The method of claim 2 wherein the human Reg protein is a recombinant protein.

5. The method of claim 3 wherein the recombinant protein is made in Pichia pastoris.

6. The method of claim 4 wherein the recombinant protein is made in Pichia pastoris.

7. The method of claim 1 wherein Reg III protein is administered.

8. The method of claim 1 wherein Reg IV protein is administered.

9. The method of claim 2 wherein Reg III protein is administered.

10. The method of claim 2 wherein Reg IV protein is administered.

11. The method of claim 7 or 9 wherein the human Reg III protein has the sequence of SEQ ID NO: 1.

12. The method of claim 8 or 10 wherein the human Reg IV protein has the sequence of SEQ ID NO: 2.

13. The method of claim 1 or 2 wherein the therapeutic agent is administered intravenously.

14. The method of claim 1 or 2 wherein the therapeutic agent is administered intrathecally.

15. The method of claim 1 or 2 wherein the therapeutic agent is administered intranasally.

16. The method of claim 1 or 2 wherein the therapeutic agent is administered transdermally.

17. The method of claim 1 or 2 wherein the therapeutic agent is administered subcutaneously.

18. The method of claim 1 or 2 wherein the therapeutic agent is administered orally.

19. The method of claim 1 wherein the disease indicator is fatigue.

20. The method of claim 1 wherein the disease indicator is difficulty of walking.

21. The method of claim 1 wherein the disease indicator is a bowel and/or bladder disturbance.

22. The method of claim 1 wherein the disease indicator is a visual problem.

23. The method of claim 1 wherein the disease indicator is a change in cognitive function.

24. The method of claim 1 wherein the disease indicator is an abnormal sensation.

25. The method of claim 1 wherein the disease indicator is a change in sexual function.

26. The method of claim 1 wherein the disease indicator is pain.

27. The method of claim 1 wherein the disease indicator is depression and/or mood swings.

28. A method for treating optic neuritis, comprising:
administering to an optic neuritis patient an effective amount of a therapeutic agent selected from the group consisting of: human Reg III, human Reg IV protein, and combinations thereof, whereby one or more clinical disease indicators of optic neuritis are reduced.

29. A method for treating neuromyelitis optica or Devic's disease, comprising:
administering to a neuromyelitis optica or Devic's disease patient an effective amount of a therapeutic agent selected from the group consisting of: human Reg III, human Reg IV protein, and combinations thereof, whereby one or more clinical disease indicators of neuromyelitis optica or Devic's disease are reduced.

30. A method for treating adrenoleukodystrophy or adrenomyeloneuropathy, comprising:
administering to an adrenoleukodystrophy or adrenomyeloneuropathy patient an effective amount of a therapeutic agent selected from the group consisting of: human Reg III, human Reg IV protein, and combinations thereof, whereby one or more clinical disease indicators of adrenoleukodystrophy or adrenomyeloneuropathy are reduced.

31. A method for treating a spinal cord injury, comprising:
administering to a spinal cord injury patient an effective amount of a therapeutic agent selected from the group consisting of: human Reg III, human Reg IV protein, and combinations thereof, whereby one or more clinical disease indicators of spinal cord injury are reduced.

32. A method for treating amyotrophic lateral sclerosis, comprising:
administering to an amyotrophic lateral sclerosis patient an effective amount of a therapeutic agent selected from the group consisting of: human Reg III, human Reg IV protein, and combinations thereof, whereby one or more clinical disease indicators of amyotrophic lateral sclerosis are reduced.

33. A method for treating inflammatory bowel disease, comprising:
administering to an inflammatory bowel disease patient an effective amount of a therapeutic agent selected from the group consisting of: human Reg III, human Reg IV protein, and combinations thereof, whereby one or more clinical disease indicators of inflammatory bowel disease are reduced.

34. A method for treating or preventing sepsis, comprising:
administering to a sepsis patient or a patient at risk of developing sepsis an effective amount of a therapeutic agent selected from the group consisting of: human Reg III, human Reg IV protein, and combinations thereof, whereby one or more clinical disease indicators of sepsis are reduced or prevented.

35. The method of claim 34 wherein the clinical disease indicator is death.

36. The method of any of claims 28-34 wherein the human Reg protein is a recombinant protein.

37. The method of claim 36 wherein the recombinant protein is made in Pichiapastoris.

38. The method of any of claims 28-34 wherein Reg III protein is administered.

39. The method of any of claims 28-34 wherein Reg IV protein is administered.

40. The method of 38 wherein the human Reg III protein has the sequence of SEQ ID NO: 1.

41. The method of claim 39 wherein the human Reg IV protein has the sequence of SEQ ID NO: 2.

42. The method of any of claims 28-34 wherein the therapeutic agent is administered intravenously.

43. The method of claim any of claims 28-34 wherein the therapeutic agent is administered intrathecally.

44. The method of claim any of claims 28-34 wherein the therapeutic agent is administered intranasally.

45. The method of claim any of claims 28-34 wherein the therapeutic agent is administered transdermally.

46. The method of claim any of claims 28-34 wherein the therapeutic agent is administered subcutaneously.

47. The method of any of claims 28-34 wherein the therapeutic agent is administered orally.

48. The method of claim 34 wherein the expected life span of the patient is extended.

49. The method of any of claims 1, 2, or 28-34 wherein between 0.1 and 10 mg/kg are administered.


SEQUENCE LISTING (10 sequences)

SEQ ID NO: 1 - protein, 175 aa, Homo sapiens
MLPPMALPSVSWMLLSCLMLLSQVQGEEPQRELPSARIRCPKGSKAYGSHCYALFLSPKSWTDADLACQKRPSGNLVSVLSGAEGSFVSSLVKSIGNSYSYVWIGLHDPTQGTEPNGEGWEWSSSDVMNYFAWERNPSTISSPGHCASLSRSTAFLRWKDYNCNVRLPYVCKFTD

SEQ ID NO: 2 - protein, 158 aa, Homo sapiens
MASRSMRLLLLLSCLAKTGVLGDIIMRPSCAPGWFYHKSNCYGYFRKLRNWSDAELECQSYGNGAHLASILSLKEASTIAEYISGYQRSQPIWIGLHDPQKRQQWQWIDGAMYLYRSWSGKSMGGNKHCAEMSSNNNFLTWSSNECNKRQHFLCKYRP

SEQ ID NO: 3 - protein, 4 aa, Homo sapiens
DIIM

SEQ ID NO: 4 - DNA, 30 bp, Homo sapiens
ggaattcgatatcatcatgagacccagctg

SEQ ID NO: 5 - DNA, 31 bp, Homo sapiens
ctaactcctgcacagccccgtcctctagagg

SEQ ID NO: 6 - protein, 20 aa, Homo sapiens
EEPQRELPSARIRCPKGSKA

SEQ ID NO: 7 - protein, 8 aa, Homo sapiens
DIIMRPSC

SEQ ID NO: 8 - protein, 10 aa, Homo sapiens
EFDIIMRPSC

SEQ ID NO: 9 - protein, 7 aa, Homo sapiens
EFDIIMR

SEQ ID NO: 10 - protein, 226 aa, Artificial Sequence
  fusion construct of engineered human Reg IV sequence and yeast alpha-factor signal sequence
MRFPSIFTAVLFAASSALAAPVNTTTEDETAQIPAEAVIGYSDLEGDFDVAVLPFSNSTFINTTIASIANNGLLAKEEGVSLEKREAEAEFDIIMRPSCAPGWFYHKSNYGYFRKLRNWSDAELECQSYGNGAHLASILSLKEASTIAEYISGYQRSQPIWIGLHDPQKRQQWQWIDGAMYLYRSWSGKSMGGNKHCAEMSSNNNFLTWSSNECNKRQHFLCKYRP